(19) United States
(12) Patent Application Publication
(10) Pub. No.: US 2025/0375235 A1
(43) Pub. Date: Dec. 11, 2025

(54) CLOSED-LOOP CRYOGENIC SYSTEMS AND PROCESSES FOR TREATING CERVICAL ABNORMALITIES

(71) Applicant: ANANYA HEALTH INC., San Francisco, CA (US)

(72) Inventors: Julie Yip, Berkeley, CA (US); Wei-Hsiang Chang, San Diego, CA (US); Anubhuti Parvatiyar, San Francisco, CA (US); Christina Skieller, Oakland, CA (US); Bruce Addis, Redwood City, CA (US); Daniel Burnett, San Francisco, CA (US); Nathan Esterkyn, Golden, CO (US); David Lehmann, Ferndale, WA (US)

(21) Appl. No.: 19/128,507

(22) PCT Filed: Nov. 7, 2023
(86) PCT No.: PCT/US2023/079017

§ 371 Date: May 8, 2025

Related U.S. Application Data
(60) Provisional application No. 63/382,686, filed on Nov. 7, 2022.

(51) Int. Cl. A61B 18/02; A61B 18/00

(52) U.S. Cl. CPC A61B 18/02; A61B 2018/00559; A61B 2018/00696; A61B 2018/0262

(57) ABSTRACT

A closed-loop cryotherapy system and related processes are described herein. In various embodiments, the system includes a front-end probe assembly and a backend cryogenic circulating unit designed to provide effective cryotherapy treatment of cervical tissue anomalies without using consumable gases or a hard-wired stable power grid.

### CROSS-REFERENCE TO RELATED APPLICATIONS

This application claims the benefit of and priority to U.S. Patent Application No. 63/382,686, filed Nov. 7, 2022, titled “SMALL SCALE REFRIGERATION SYSTEMS AND PROCESSES,” the disclosure of which is incorporated herein by reference in its entirety.

### TECHNICAL FIELD

The present systems and processes relate generally to cryogenic treatment of a lesion of the cervix or similar area.

### BACKGROUND

Every two minutes, a woman dies of cervical cancer, and cervical cancer is the fourth most common cancer in women. While cervical cancer deaths have dramatically fallen in high-income countries, cervical cancer is a leading cause of cancer deaths for women in low- and middle-income countries (LMICs). Treatments for precancerous lesions are highly effective for preventing disease progression, so identification and treatment of lesions early is important. In high-income countries, loop electrosurgical excision procedure (LEEP) is generally considered the standard of care. However, LEEP uses expensive equipment, reliable electricity, local anesthesia, and a licensed medical doctor to perform the procedure, making LEEP inaccessible for many women in LMICs.

Cryotherapy has advantages over the LEEP procedure, particularly in that it is a short procedure (e.g., <15 minutes), can be administered by nonphysicians after minimal training, and is a relatively painless procedure without requiring anesthesia. In cryotherapy, a metallic probe is placed against the cervix and cooled to cryogenic temperatures to freeze the probe-contracting cervical tissue, causing cellular necrosis of the tissue abnormality. However, despite its advantages, current cryotherapy systems and other ablative technologies are not well-suited for LMICs in that they rely on consumable gases and steady electricity. Compressed gas is largely unavailable in many areas of LMICs and the tanks are bulky, heavy, and difficult to transport. Further, some existing cryotherapy solutions require connection to a stable power grid and are expensive. Thermal ablation can be used for treatment but is typically unable to reach a tissue ablation depth of 5 mm (the depth of effective ablation of the precancerous tissue according to the World Health Organization (WHO)). Thermal ablation is also more painful than cryotherapy.

Therefore, there is an unmet need for an effective cryogenic treatment system and method when there is limited access to external compressed gas supplies, a hard-wired power supply, or a stable power grid.

### BRIEF SUMMARY OF THE DISCLOSURE

Briefly described, and according to one embodiment, aspects of the present disclosure generally relate to systems and methods for cryogenic treatment of the cervix of the uterus or other tissues. In at least one embodiment, the system includes a medical device provided in the form of a portable, battery-powered, closed-loop cryogenic system for cryoablation treatment of cervical tissue anomalies without needing consumable gas or cryogen inputs.

According to particular embodiments, the closed-loop cryotherapy system may be applied as a treatment by a medical service provider. In some embodiments, the one or more systems and processes leverage the closed-loop cryotherapy system with a cryogenic circulating unit to provide focused cooling to a probe tip, which can be applied to freeze cervical tissue to treat cervical abnormalities, such as precancerous lesions. In some embodiments, the treatment application process can include a double freeze cycle.

According to a first aspect, the present disclosure includes a closed-loop cryotherapy system comprising: a probe assembly configured for focused cooling, the probe assembly comprising: a cervix-contacting probe tip at a distal end of the probe assembly; an internal probe body in fluid connection with the cervix-contacting probe tip and comprising an internal evaporation chamber configured to circulate one or more cryogenic materials; a cryogenic circulating unit designed to recycle the one or more cryogenic materials, the cryogenic circulating unit comprising: a compressor configured to receive the one or more cryogenic materials from the probe assembly and pressurize the one or more cryogenic materials; a condenser unit configured to convert the pressurized one or more cryogenic materials to a liquid, wherein the liquid is circulated to the probe to facilitate freezing of a precancerous lesion; a power module comprising a rechargeable battery, wherein the power module allows the closed-loop cryotherapy system to operate without a stable power grid; and a controller configured to modulate a speed of the compressor in order to adjust a freezing temperature of the probe.

In a second aspect of the closed-loop cryotherapy system of the first aspect or any other aspect, wherein the freezing temperature of the probe assembly is within −30 degrees C. and −80 degrees C.

In a third aspect of the closed-loop cryotherapy system of the second aspect or any other aspect, further comprising one or more sensors configured to determine one or more parameters of the system.

In a fourth aspect of the closed-loop cryotherapy system of the third aspect or any other aspect, wherein the one or more sensors includes a pressure sensor, a temperature sensor, or a combination thereof.

In a fifth aspect of the closed-loop cryotherapy system of the fourth aspect or any other aspect, wherein the controller modulates the speed of the compressor based on an ambient temperature of one or more aspects of the probe assembly.

In a sixth aspect of the closed-loop cryotherapy system of the fifth aspect or any other aspect, wherein the cryogenic circulating unit is coupled to the probe with a coaxial hose comprising a liquid line housed within a vapor return line to facilitate efficient freezing of the cervix-contacting probe tip.

In a seventh aspect of the closed-loop cryotherapy system of the sixth aspect or any other aspect, further comprising a throttling expansion valve configured to open and close to throttle the controller based on a pressure of the liquid in the probe compared to a setpoint threshold. In an eighth aspect of the closed-loop cryotherapy system of the seventh aspect or any other aspect, a housing configured enclose the closed-loop cryotherapy system to allow the closed-loop cryotherapy system to be portable.

In a ninth aspect of the closed-loop cryotherapy system of the eighth aspect or any other aspect, wherein the cryogenic circulating unit operates without the use of consumable gases.

According to a tenth aspect, a closed-loop cryotherapy process comprising: providing a handheld probe with a cervix-contacting probe tip configured for focused freezing of a tissue, wherein the probe assembly includes an interior chamber configured to circulate one or more cryogenic materials; recycling the one or more cryogenic materials using a cryogenic circulating unit comprising a compressor, a condenser, a power module, and a controller by: receiving the one or more cryogenic materials from the probe assembly; pressurizing the one or more cryogenic materials using the compressor; converting the one or more cryogenic materials to a liquid using the condenser unit; and circulating the liquid to the probe assembly using one or more hoses.

In an eleventh aspect of the closed-loop cryotherapy process of the tenth aspect or any other aspect, further comprising: decreasing a pressure of the liquid before circulating the liquid to the probe assembly by using an expansion valve in line with the one or more hoses.

In a twelfth aspect of the closed-loop cryotherapy process of the eleventh aspect or any other aspect, further comprising: throttling the controller by: receiving a pressure of the liquid in the probe; determining whether the pressure is above a threshold setpoint; closing a throttling expansion valve when the pressure is greater than the threshold setpoint; and opening the throttling expansion valve when the pressure is less than the threshold setpoint.

In a thirteenth aspect of the closed-loop cryotherapy process of the twelfth aspect or any other aspect, wherein the focused freezing of the tissue comprises freezing the tissue to achieve a tissue freeze radial dimension of approximately 5 mm.

In a fourteenth aspect of the closed-loop cryotherapy process of the thirteenth aspect or any other aspect, wherein the cryogenic circulating unit is configured to recycle the one or more cryogenic materials rather than venting the one or more cryogenic materials into an environment.

According to a fifteenth aspect, a closed-loop cryotherapy comprising: a probe assembly configured for freezing cervical tissue, the probe assembly comprising: a cervix-contacting probe tip at a distal end of the probe assembly; an internal probe body with an internal evaporation chamber configured to circulate one or more cryogenic materials; a cryogenic circulating unit designed to capture the one or more cryogenic materials the cryogenic circulating unit comprising: a compressor configured to receive the one or more cryogenic materials from the probe assembly via a vapor return line and pressurize the one or more cryogenic materials; an oil separator loop configured to process the one or more cryogenic materials; a condenser unit configured to convert the one or more cryogenic materials from the oil separator loop to a liquid; a capillary tube configured to carry the liquid to the cervix-contacting probe tip; an expansion valve in line with the capillary tube designed to generate an asymmetric pressure; a power module comprising a battery, wherein the power module allows the closed-loop cryotherapy system to operate without a stable power grid; and a controller configured to modulate a speed of the compressor in order to adjust a freezing temperature of the probe assembly.

In a sixteenth aspect of the closed-loop cryotherapy system of the fifteenth aspect or any other aspect, wherein the freezing temperature of the cervix-contacting probe tip is within −30 degrees C. and −80 degrees C.

In a seventeenth aspect of the closed-loop cryotherapy system of the sixteenth aspect or any other aspect, wherein the cervix-contacting probe tip is used to freeze a precancerous lesion by applying the cervix-contacting probe tip to an area of tissue until a tissue freeze radial dimension of 5 mm is achieved.

According to an eighteenth aspect, a method for treating precancerous lesions of a cervix using a closed-loop cryotherapy system, the method comprising: positioning a portable housing containing the closed-loop cryotherapy system near a patient, wherein the closed-loop cryotherapy system is configured with a rechargeable power module to allow the closed-loop cryotherapy system to operate without a stable power grid and a cryogenic circulating unit designed to recycle one or more cryogenic materials to allow the closed-loop cryotherapy system to operate without compressed gas; initiating focused cooling of a probe assembly of the closed-loop cryotherapy system, wherein the probe assembly comprises a cervix-contacting probe tip at a distal end of the probe assembly; determining the cervix-contacting probe tip has a temperature within −30 degrees C. and −80 degrees C.; and applying the cervix-contacting probe tip to the cervix until a tissue freeze radial dimension of 5 mm is achieved.

According to a nineteenth aspect of the method of the eighteenth aspect, or any other aspect, further comprising: throttling a controller of the closed-loop cryotherapy system by: receiving a pressure of a liquid in the probe; determining whether the pressure is above a threshold setpoint; closing a throttling expansion valve when the pressure is greater than the threshold setpoint; and opening the throttling expansion valve when the pressure is less than the threshold setpoint.

According to a twentieth aspect of the method of the nineteenth aspect, or any other aspect, further comprising: allowing the closed-loop cryotherapy system to operate for at least 10 minutes before applying the cervix-contacting probe tip to the cervix.

According to a twenty-first aspect, the system includes refrigeration system comprising: a probe assembly operatively connected to a cryogenic circulating unit designed to recycle cryogenic material, the probe assembly comprising: a freezing probe tip at a distal end of the probe assembly; and an internal probe body in fluid connection with the freezing probe tip and comprising an internal evaporation chamber configured to circulate one or more cryogenic materials, wherein: the probe receives the one more cryogenic materials in a liquid form from a condenser unit of the cryogenic circulating unit thereby cooling the freezing probe tip to −30° C. to −80° C.; the freezing probe tip is configured to contact human or animal anatomy and absorb heat therefrom, thereby converting the one or more cryogenic materials into a gas form via the internal evaporation chamber and cooling the human or animal anatomy; and the probe assembly transfers the one or more cryogenic materials in the gas form to the cryogenic circulating unit for pressurizing, condensing, and recycling the one or more cryogenic materials.

According to a twenty-second aspect, the system includes a refrigeration system comprising: a cryogenic circulating unit designed to recycle one or more cryogenic materials comprising: a power module comprising a rechargeable battery, wherein the power module enables the cryogenic circulating unit to operate without a stable power grid; a controller configured to modulate a speed of a compressor in order to adjust a temperature of the one or more cryogenic materials; the compressor configured to receive the one or more cryogenic materials from a probe assembly in a gas form and pressurize the one or more cryogenic materials; and a condenser unit configured to convert the pressurized one or more cryogenic materials to a liquid, wherein the liquid is circulated to the probe assembly at a temperature at a probe tip of the probe assembly of −30° C. to −80° C. to facilitate freezing of a precancerous lesion.

These and other aspects, features, and benefits of the systems and processes described herein will become apparent from the following detailed written description taken in conjunction with the following drawings, although variations and modifications thereto may be affected without departing from the spirit and scope of the novel concepts of the disclosure.

### BRIEF DESCRIPTION OF THE DRAWINGS

The accompanying drawings illustrate one or more embodiments and/or aspects of the disclosure and, together with the written description, serve to explain the principles of the disclosure. Wherever possible, the same reference numbers are used throughout the drawings to refer to the same or like elements of an embodiment, and wherein:

FIG. 1 illustrates a system diagram of the closed-loop cryotherapy system according to one embodiment of the present disclosure.

FIG. 2 illustrates an exemplary application of a probe tip of the closed-loop cryotherapy system to a cross-section of a cervix according to one embodiment of the present disclosure.

FIG. 3 illustrates an exemplary probe assembly of the closed-loop cryotherapy system according to one embodiment of the present disclosure.

FIG. 4A illustrates an exemplary probe assembly of the closed-loop cryotherapy system according to one embodiment of the present disclosure.

FIG. 4B illustrates an exemplary probe assembly of the closed-loop cryotherapy system according to one embodiment of the present disclosure.

FIG. 4C illustrates an exemplary probe assembly of the closed-loop cryotherapy system according to one embodiment of the present disclosure.

FIG. 4D illustrates an exemplary probe assembly of the closed-loop cryotherapy system according to one embodiment of the present disclosure.

FIG. 4E illustrates an exemplary probe assembly of the closed-loop cryotherapy system according to one embodiment of the present disclosure.

FIG. 4F illustrates an exemplary probe assembly of the closed-loop cryotherapy system according to one embodiment of the present disclosure.

FIG. 5A illustrates an exemplary probe assembly of the closed-loop cryotherapy system according to one embodiment of the present disclosure.

FIG. 5B illustrates a cross-section of one embodiment of the exemplary probe assembly of FIG. 5A according to one embodiment of the present disclosure.

FIG. 5C illustrates a cross-section of one embodiment of the exemplary probe assembly of FIG. 5A according to one embodiment of the present disclosure.

FIG. 6A illustrates an exemplary probe assembly of the closed-loop cryotherapy system according to one embodiment of the present disclosure.

FIG. 6B illustrates an exemplary probe assembly of the closed-loop cryotherapy system according to one embodiment of the present disclosure.

FIG. 7A illustrates an exemplary probe assembly of the closed-loop cryotherapy system according to one embodiment of the present disclosure.

FIG. 7B illustrates an exemplary probe assembly of the closed-loop cryotherapy system according to one embodiment of the present disclosure.

FIG. 8 is a flow diagram of a throttling process to modulate the flow of cryogenic materials through the closed-loop cryotherapy system according to one embodiment of the present disclosure.

FIG. 9 illustrates an exemplary medical device for applying the closed-loop cryotherapy system as a treatment for cervical anomalies according to one embodiment of the present disclosure.

FIG. 10 is a flow diagram of a cryotherapy process for activating a double freeze treatment cycle according to one embodiment of the present disclosure.

FIG. 11 is a flow diagram of a cryoablation process for applying a double freeze treatment cycle to treat a precancerous lesion or other tissue abnormality to one embodiment of the present disclosure.

While the disclosure is susceptible to various modifications and alternative forms, specific embodiments thereof are shown by way of example in the drawings and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure.

### DETAILED DESCRIPTION

To promote an understanding of the principles of the present disclosure, reference will now be made to the embodiments illustrated in the drawings and specific language will be used to describe the same. It will, nevertheless, be understood that no limitation of the scope of the disclosure is thereby intended; any alterations and further modifications of the described or illustrated embodiments, and any further applications of the principles of the disclosure as illustrated therein are contemplated as would normally occur to one skilled in the art to which the disclosure relates. All limitations of scope should be determined by and as expressed in the claims.

Whether a term is capitalized is not considered definitive or limiting of the meaning of a term. As used in this document, a capitalized term shall have the same meaning as an uncapitalized term, unless the context of the usage specifically indicates that a more restrictive meaning for the capitalized term is intended. However, the capitalization or lack thereof within the remainder of this document is not intended to be necessarily limiting unless the context clearly indicates that such limitation is intended.

### Overview

In various embodiments, aspects of the present disclosure generally relate to systems and processes for cryotherapy treatment of cervical anomalies using a medical device with a closed-loop cryotherapy system that recycles the cryogenic materials used to provide focused cooling to an ergonomic probe assembly. In particular embodiments, probe assembly includes a probe tip, which can be applied to an area of cervical tissue to achieve a desired freeze zone for effective cryotherapy treatment of precancerous lesions. The treatment device and system disclosed herein can also be battery-operated, portable, and durable. As a result, the systems and processes described herein increase the ability to treat patients in remote areas without access to a stable power supply and/or consumable gases.

### Description of the Figures

For the purposes of example and explanation of the fundamental processes and components of the disclosed systems and processes, reference is made to the figures. In general, the figures illustrate a closed-loop cryotherapy medical device system processes for operating such system to provide cryotherapy treatment.

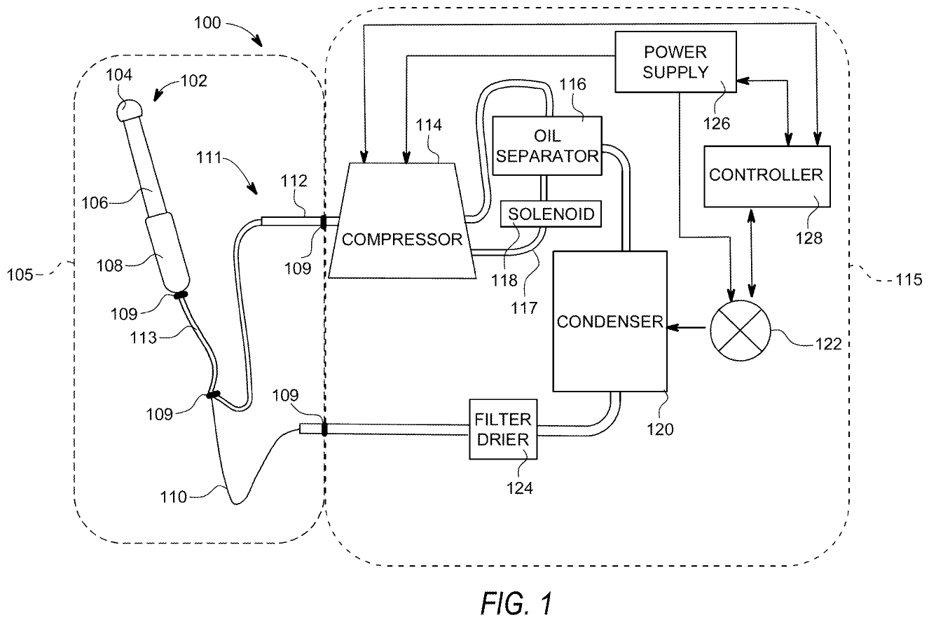

FIG. 1 illustrates a non-limiting example of a closed-loop cryotherapy system 100 according to embodiments of the present disclosure. Various aspects of the embodiment shown in FIG. 1 include a two-section refrigeration system, the first section comprising a patient-facing front-end subsystem 105 and a second section comprising a cryogenic circulating unit backend subsystem 115 (referred to herein as a cryogenic circulating unit 115) of the closed-loop cryotherapy system 100. It will be appreciated that where used herein, the “closed-loop” cryotherapy system refers to a system that recycles and/or reuses the materials used in the freezing process(es), instead of venting the used cryogenic materials and relying an external compressed gas supply as a source of new refrigerant for the process cycles.

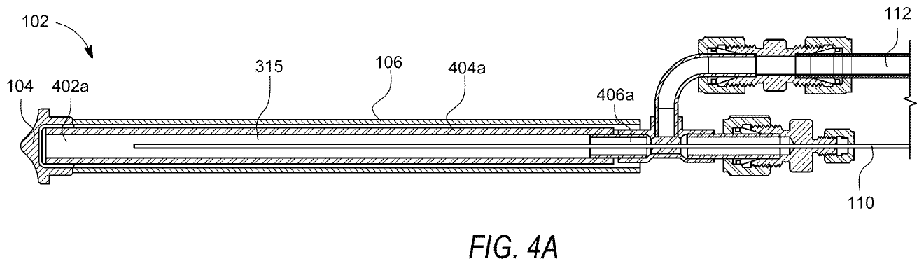

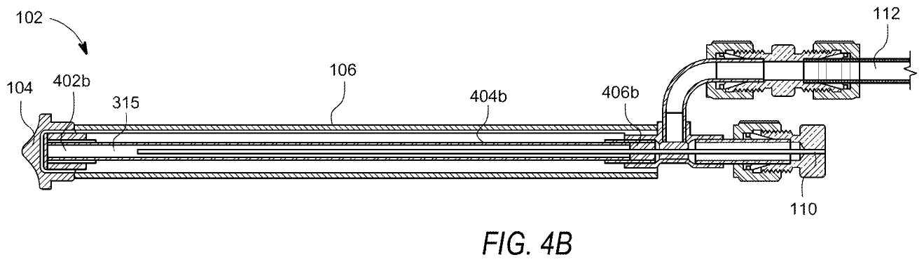

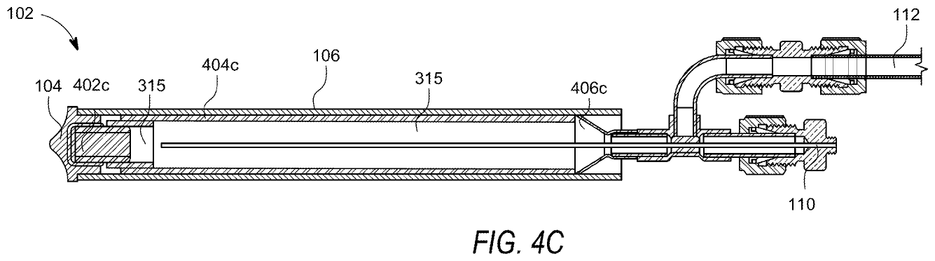

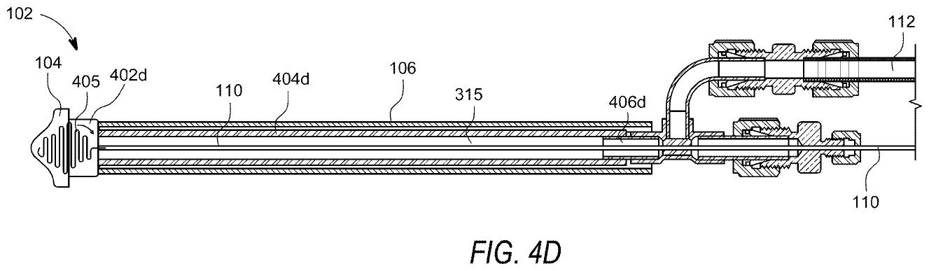

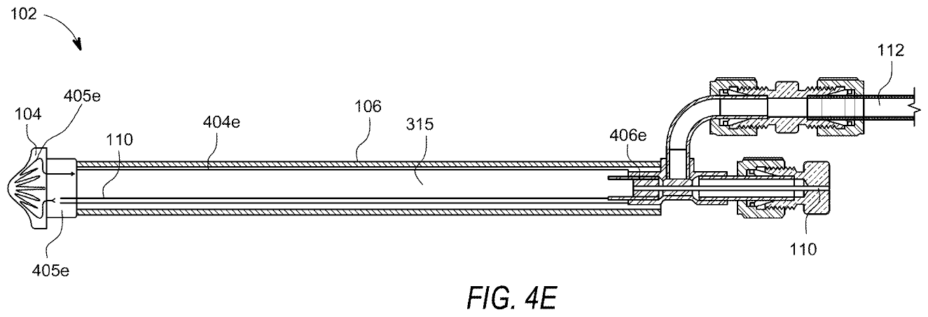

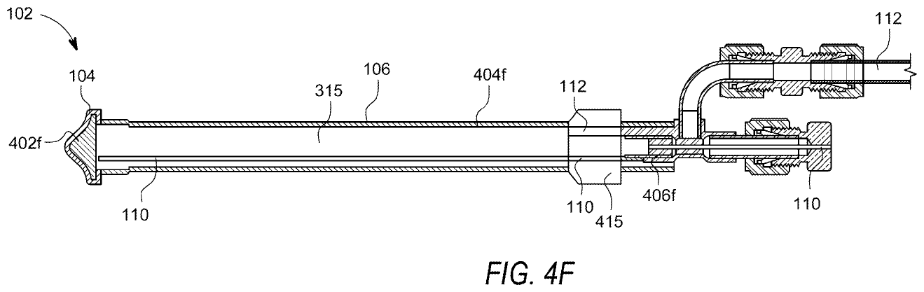

In some embodiments, the front-end subsystem 105 includes a probe assembly 102 and a hose assembly 111. In particular embodiments, the probe assembly 102 includes a cervix-contacting probe tip 104, a probe sheath 106, and a handle 108. In some embodiments, the probe sheath 106 is provided in the form of an outer plastic housing that encloses a metal internal probe body (see FIGS. 4A-7B), wherein the internal probe body is brazed to, and in fluid connection with, the probe tip 104 and coupled to the handle 108. In some forms, the probe assembly 102 is operatively coupled to the cryogenic circulating unit 115 via the hose assembly 111. In some embodiments, the hose assembly 111 can include a liquid line inlet 110 and a vapor return line 112. In various embodiments, the liquid line inlet 110 and the vapor return line 112 are enclosed in a single hose 113. In some embodiments, the hose 113 can contain the liquid line inlet 110 and the vapor return line 112 from the backend cryogenic circulating unit 115 to the probe assembly 102. It will be appreciated that although FIG. 1 shows separate connections at the backend subsystem 115 and the front-end subsystem 105 for the inlet liquid line 110 and the vapor return line 112, this can be provided in the form of a single connection point, for connecting the hose 113 of the hose assembly 111. In some embodiments, the probe assembly 102 is coupled to a distal end of the hose assembly 111, wherein the proximal end of the hose assembly 111 is coupled to the cryogenic circulating unit 115. In some embodiments, a quick connection 109 can be located at one or more locations of the front-end subsystem 105, including but not limited to the proximal end of the probe assembly 102, within the hose assembly 111, at the connection point(s) between the front-end subsystem 105 and the backend subsystem 115, or a combination thereof.

In the embodiment shown in FIG. 1, the liquid line inlet 110 is configured to transport a cryogenic material in a liquid form to the probe assembly 102 and the vapor return line 112 is configured to transport the cryogenic material in a vapor form from the probe assembly 102 to the backend cryogenic circulating unit 115 to be recycled. In this embodiment, the cryogenic materials are pressurized in a liquid form and expand to a gas when the liquid refrigerant reaches the probe assembly 102, such that the cryogenic materials expand into a vapor at the probe tip 104 and the probe tip 104 is cooled to the desired freezing temperature(s).

The liquid line inlet 110 and the vapor return line 112 can be provided in the form of pressure-rated and refrigerant-compatible hoses. In one non-limiting embodiment, the vapor return line 112 can be provided in the form of a coaxial, nylon, or refrigerant hose. In various embodiments, the hose 113 is provided in the form of a burst-proof hose sheath to house the liquid line inlet 110 and vapor return line 112 between the cryogenic circulating unit 115 and the probe assembly 102. In one embodiment, the liquid line inlet 110 is housed within the larger diameter vapor return line 112. In some embodiments, the liquid line inlet is run through the center of the vapor return line 112 or otherwise housed in a multi-lumen configuration. In this example, the liquid line inlet 110 liquid line is protected from bends and kinks within the walls of the vapor return line 112. As discussed in more detail below in FIGS. 4A-7B, the size of the liquid line inlet 110 can be sized relative to the volume of the probe sheath 106 to optimize the focused cooling of the probe tip 104 to achieve a specific freeze depth, freeze time, freeze temperature, or a combination thereof.

Where used throughout, “cryogenic materials,” can refer to one or more refrigerants or coolants in various phase states (e.g., liquid, vapor, gas, gel, etc.). According to some embodiments, the cryogenic materials have a low global warming potential (GWP), have zero ozone depletion potential (ODP), are suitable for low and middle back pressure compressor systems, and have a dew point or boiling point below 10° C. at atmospheric pressure. In some embodiments, the one or more cryogenic materials can include, but is not limited to one or more of the following refrigerants or coolants:

| | Trade or Common | |
|---|---|---|
| Coolant Name | Name | CAS Name |
| R-717 | Ammonia | Ammonia |
| R-744 | CO2 | Carbon dioxide |
| R-744A | N2O | Nitrous Oxide |
| R-290 | Propane | Propane |
| R-600a | Isobutane | Isobutane |
| R-170 | Ethane | Ethane |
| R-601 | Pentane | Pentane |
| R-161 | HFC-161 | Fluoroethane |
| R-152a | HFC-152a | 1,1-Difluoroethane |
| R-32 | HFC-32 | Difluoromethane |
| R-365mfc | HFC-365mfc | 1,1,1,3,3-Pentafluorobutane |
| R-245fa | HFC-245fa | 1,1,1,3,3-Pentafluoropropane |
| R-416A | | R-134a/R-124/R-600 (59/39.5/1.5) |
| R-414B | Hot Shot | (50/39/1.5/9.5) |
| R-449 | Opteon XP40 | R-32/R-125/R-1234yf/R-134a (24.3/24.7/25.3/25.7) |
| R-134a | HFC-134a | 1,1,1,2-Tetrafluoroethane |
| R-4310mee | HFC-43-10mee, HFC- | decafluoropentane |
| | 4310mee, R-43-10mee | |
| R-407C | | R-32/R-125/R-134a (23/25/52) |
| R-437A | MO49 Plus | R-32/R-125/R-600a/R-601 (78.5/19.5/1.4/0.6) |
| R-407F | | R-134a/R-125/R-32 (40/30/30) |
| R-413A | MO49 | R-218/R-134a/R-600a (9/88/3) |
| R-410A | Puron, AZ-20 | R-32/R-125 (50/50) |
| R-407A | KLEA 60 | R-32/R-125/R-134a (20/40/40) |
| R-427A | | R-32/R-125/R-143a/R-134a (15/25/10/50) |
| R-438A | MO99 | R-125/R-134a/R-32/R-600a (45/44.2/8.5/2.3) |
| R-423A | 39TC | R-134a/R-227ea (52.5/47.5) |
| R-417A | MO59, NU22 | R-125/R-134a/R-600 (46.6/50.0/3.4) |
| R-424A | RS-44 | R-125/R-134a/R-600a/R-600/R-601a (50.5/47/.9/1/.6) |
| R-422B | NU-22B | R-125/R-134a/R-600a (55/42/3) |
| R-421A | | R-125/R-134a (58/42) |
| R-422D | MO29 | R-125/R-134a/R-600a (65.1/31.5/3.4) |
| R-407B | | R-32/R-125/R-134a (10/70/20) |
| R-422C | One Shot | R-125/R-134a/R-600a (82/15/3) |
| R-422A | | R-125/R-134a/R-600a (85.1/11.5/3.4) |
| R-227ea | HFC-227ea | 1,1,1,2,3,3,3-Heptafluoropropane |
| R-125 | HFC-125 | Pentafluoroethane |
| Isceon MO89 | | R-125/R-218/R-290 (86/9/5) |
| R-404A | HP-62 | R-125/R-143a/R-134a (44/52/4) |
| R-507 | AZ-50 | R-125/R-143a (50/50) |
| R-143a | HFC-143a | 1,1,1-Trifluoroethane |
| EP-88 | | |
| R-13b1 | Halon 1301 | Bromotrifluoromethane |
| R-14 | PFC-14, CF4 | Tetrafluoromethane |
| R-500 | | R-12/R-152a (73.8/26.2) |
| R-218 | PFC-218 | Octafluoropropane |
| R-236fa | HFC-236fa | 1,1, 1,3,3,3-Hexafluoropropane |
| R-116 | PFC-116 | Hexafluoroethane |
| R-508B | | R-23/R-116 (46/54) |
| R-503 | | R-23/R-13 (40.1/59.9) |
| R-23 | HFC-23 | Trifluoromethane |

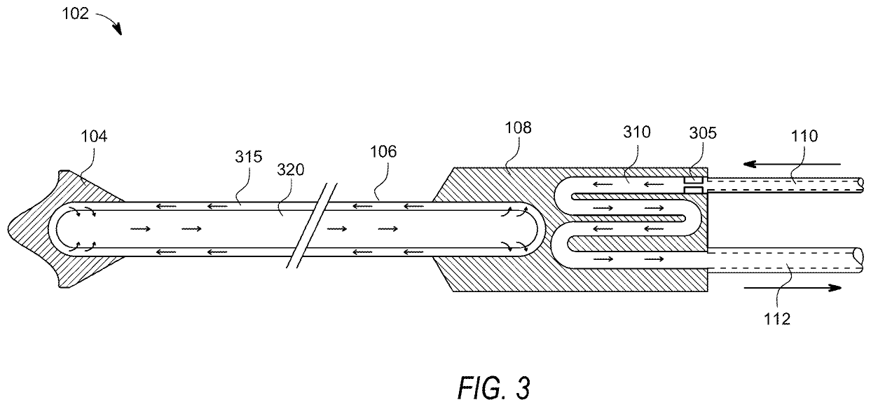

The exemplary probe assembly 102 can include one or more internal evaporation channels 315 (see FIG. 3) or channels to facilitate the state change of the cryogenic materials to cool the probe tip 104. In some embodiments, the probe sheath 106 houses the one or more internal evaporation champers. In some embodiments, the probe assembly 102 can include one or more evaporation coils. In one or more embodiments, the one or more evaporation coils can be located in the probe tip 104, in the probe sheath 106, or a combination thereof. In some embodiments, the handle 108 can be provided in the form of an ergonomic handle. In a non-limiting exemplary embodiment, the handle 108 can be provided in the form of an ergonomic pistol-grip handle wand. In some embodiments, the form of the handle 108 can be tailored for multiple hand sizes and orientations (e.g., left-hand v. right-hand). In various embodiments, the first section of the closed-loop cryotherapy system is an advanced probe assembly 102 designed to work in connection with the second section comprising the cryogenic circulating unit 115, wherein circulated cryogenic materials travel within an internal chamber of the probe assembly (see FIGS. 4A-7B) to generate freezing temperatures at the probe tip 104, which can be applied to a patient to treat one or more tissue anomalies.

In an exemplary embodiment, the cryogenic circulating unit 115 is a connected system for recycling the cryogenic materials used to cool the probe tip 104. In at least this way, the closed-loop cryotherapy system 100 operates without the use of consumable gases, like an external compressed supply of nitrogen (N₂) or carbon dioxide (CO₂) gases. Further, the closed-loop cryotherapy system 100 recycles the cryogenic materials using the cryogenic circulating unit 115 rather than venting the cryogenic materials into the environment, thereby reducing and/or eliminating harmful emissions.

The cryogenic circulating unit 115 can include, but is not limited to, a compressor 114, an oil separator 116, a condenser unit 120, a fan 122, a filter drier 124, a power module 126, and a controller 128. In at least one embodiment, the compressor 114 is configured to receive gaseous cryogenic materials (e.g., refrigerant) from the vapor return line 112 of the probe assembly 102. In particular embodiments, the compressor 114 performs mechanical operations to pressurize the cryogenic material. In some embodiments, the compressor 114 is provided in the form of a single-stage compressor unit. According to embodiments, the compressor has a displacement range of approximately 1 cm³ to 5 cm³ and/or a cooling capacity range of approximately 30 Watts to 1000 Watts. In various embodiments, the compressor 114 has one or more of the following features: variable-speed efficiency, low-noise, accommodates refrigerants with a low GWP, protection against electromagnetic interference, customized controller configurations, low back pressure, and/or a 12/24 VDC power connection. According to one embodiment, the oil separator loop includes a coalescent oil separator 116 and uses a tube 117 and a solenoid 118 to control and regulate the flow of cryogenic materials back into the compressor. In some embodiments, the solenoid 118 is provided in the form of a ball valve. In one non-limiting example, the oil separator loop is opened for a first time period by opening the solenoid 118 before closing the oil separator loop by closing the solenoid 118. The exemplary process of closing the solenoid 188 decreases flow through the oil separator 116, which creates a low-pressure vacuum in the compressor 114, In various embodiments, the tube 117 regulates and decreases the flow of the oil into the compressor 114. According to some embodiments, the oil separator 116 is at least 90% efficient in removing particulates below 5 μm and/or is not dependent on velocity for efficiency and maintains consistency down to 20% of the maximum flow rate.

Once pressurized, the cryogenic material from the compressor 114 passes to the condenser unit 120, which is configured to initiate a phase change of the cryogenic material from a vapor to a liquid before returning to the probe assembly 102 to facilitate freezing of a precancerous lesion. In some embodiments, the cryogenic material from the compressor 114 is superheated vapor which is routed to the condenser 120 unit to transform the cryogenic material into pressurized liquid. In some embodiments, the condenser unit 120 utilizes a fan 122 to remove heat from the cryogenic material and enhance the state-change process. In some embodiments, the fan 122 can be integral to the condenser unit 120. According to some embodiments, the fan 122 has variable speed efficiency and can have an operating range of approximately 30 cubic feet per minute (CFM) to 300 CFM. In various embodiments, the condenser unit 120 can include one of a plurality of coil orientations (e.g., vertical, flat, stationary, constant motion, single tube, serpentine, etc.). In some embodiments, the condenser unit 120 may be comprised of fluid coils and/or micro coils. In some embodiments, the system includes a water, gel, or ice-bath cooled condenser unit 120 to further decrease the temperature of the cryogenic materials. In various embodiments, as the pressurized liquid cryogenic material leaves the condenser unit 120, the filter drier 124 is used to filter any contaminants and absorb any moisture (e.g., water) from the liquid line before the liquid is carried to the probe assembly 102 for focused cooling. According to some embodiments, the filter drier 124 can be provided in the form of solid sieve core copper with a steel outer body or a sintered spun copper with a copper outer body. In some embodiments, the filter drier 124 is sized to match the outer diameter of the liquid line inlet 110. In some embodiments, the system can further include a sight glass (not shown) in connection with one or more aspects of the front-end subsystem 105 or the cryogenic circulating unit 115 to provide a visual confirmation of the phase-state change.

The power module 126 is used to provide electrical power to the system components to allow the cryogenic circulating unit 115 to continuously circulate the cryogenic materials for the duration of a treatment procedure. In some embodiments, the power module 126 can provided in the form of a rechargeable 12V lithium iron phosphate (LiFePO₄) battery. In an exemplary embodiment, the LiFePO4 battery provides electrical power that is less prone to thermal runaway and combustion than traditional lithium-ion batteries and can provide an improved life cycle over traditional lithium-ion and lead acid batteries. It will be appreciated that the power module 126 can be provided in the form of other types of batteries (e.g., different voltages, different battery chemistries, physical size differences, etc.), alternative power sources (e.g., solar power, hydropower, wind power, generator, portable power bank, super capacitor, etc.), or a combination thereof. In some embodiments, the system can be provided with a rechargeable battery-powered power module 126 (or other type of portable power supply) in addition to a hard-wired power module 126, to provide additional accessibility for locations that may or may not have access to a stable power grid. In some embodiments, the power module 126 can be provided in the form of a hard-wired power supply or a standard 120V plug-in connection (or other standard voltage according to various international standards). In some embodiments, the power module 126 can be provided in the form of one or more hot-swappable batteries. In this non-limiting example, the system can be configured with a battery module configured to accept standard-sized batteries (e.g., 12 VDC of various capacities, 7 Ah, 20 Ah, etc.). It will be appreciated that other voltages and battery configurations are contemplated within the scope of the present disclosure. In various embodiments, the system can further include a battery management system for onboard charging and/or predictive analytics related to battery charge (e.g., X number of treatments remaining on the current battery).

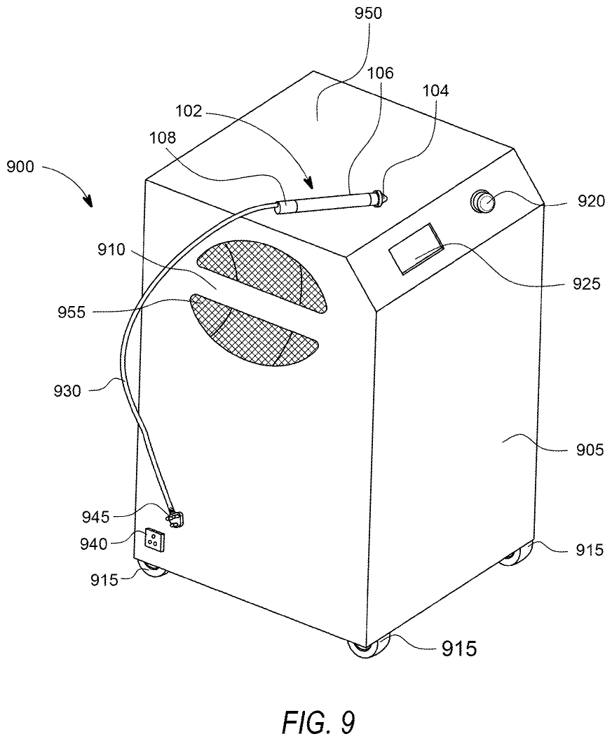

In some embodiments, the controller 128 can be provided in the form of an onboard control system for monitoring and controlling the closed-loop cryogenic system 100. In some embodiments, the controller 128 can be provided in the form of a data-processing device configured to transmit and receive data sets from one or more of the components of the system 100. The controller 128 can also include one or more sensor modules, or other hardware components designed to facilitate the monitoring and control of one or more of the components of the system 100. In one non-limiting example, the controller 128 can include a pressure sensor for detecting the pressure of the system 100 or a specific subsystem or component of the system 100. In another non-limiting example, the controller 128 can include a temperature sensor for detecting the temperature of an aspect of the system 100. In some embodiments, the temperature sensor can be used to determine the temperature of one or more portions of the probe assembly 102, the temperature of the cryogenic materials at each stage of the cryogenic recycling process (e.g., start of the vapor return line 112 at the probe assembly 102, the suction temperature at the inlet of the compressor 114 and the discharge temperature, the end of the liquid line inlet 110 at the probe assembly 102, etc.), the internal temperature of the housing 905 (see FIG. 9), and or external ambient temperature of the environment, or a combination thereof. In some embodiments, the system can include both a pressure sensor and a temperature sensor. In this non-limiting example, the controller 128 can regulate the speed of the compressor 114 and/or the condenser fan 122 to increase or decrease the pressure and/or temperature of the cryogenic materials, based on the information obtained from the pressure sensor and/or temperature sensor. It will be appreciated that other sensors can be included in some configurations of the system described herein. In various embodiments, the system may include an attachment module (not shown) that can be used as an optional secondary device to the main system. In some embodiments, the attachment module may include a multi-use disposable device monitoring attachment or similar add-on device for sensing system parameters (e.g., temperature, pressure, voltage, etc.). The attachment module can be battery-powered and functional for a specified number of treatment cycles (e.g., 10, 20, 50, etc.). The attachment module can include data recording and communications built-in to transmit the treatment session data. The attachment module can be used as a failsafe to the main system. For example, if the temperature or the pressure falls out of the specified range such that incomplete or insufficient treatment would be provided, the attachment module can have the ability to communicate with the main system to shut off the main system and inform the user of recommended maintenance steps. In some embodiments, the processes and functions of the optional attachment module can be performed by the controller 128 of the cryogenic circulating unit 115.

In some forms, the system further includes a user device in communication with the controller 128 via a network, wherein the user device can be configured to send and receive information from the closed-loop system. In some forms, the system also includes a notification module designed to generate and distribute one or more notifications to a user device or a display (either remote or onboard the system) via the controller 128. In some embodiments, the system provides monitoring and control of each of the individual system components and can communicate the component data to a remote maintenance system for efficient identification of maintenance, service, or other issues, including automatic deployment of service requests or troubleshooting steps. In at least this way, the system can provide advanced analytics for identifying a status or error code of one or more components of the system 100 and facilitate the efficient data processing to address identified issues or implement corrective action. In some embodiments, one or more advanced artificial intelligence models can be used to facilitate the data collection and/or processing steps.

It will be appreciated that FIG. 1 is only a non-limiting embodiment and configurations, subsystems, modules, components, and other aspects of a closed-loop cryotherapy system 100 can be provided according to the system and processes described herein. In one non-limiting example, the system also includes a recuperation module (not shown), which harnesses excess cooling power after the treatment application using the probe assembly 102. In this example, the recuperation module “precools” the cryogenic materials by providing additional cooling to the cryogenic materials prior to reaching the probe tip 104. In some embodiments, the recuperation module can be located in the liquid line prior to the liquid line inlet 110 and/or within the interior evaporation chamber 315 (see FIGS. 3-7B) of the probe assembly 102. In some embodiments, the system can include a secondary evaporator (not shown) after the vapor return line (i.e., vapor return line 112) to ensure complete evaporation, harness excess cooling energy, and other functions. In these non-limiting examples, providing colder incoming cryogenic materials, including the incoming liquid refrigerant, leads to improved evaporation and a colder probe tip 104 temperature.

Some embodiments can include modular components that can be interchanged. For example, the system may utilize swappable components (e.g., probe assembly 102, compressor 114, power module 126, condenser 120, filter drier 124, etc.). In at least this way, in some embodiments, the closed-loop system and associated components can be modularized, such that the system is configured to provide enhanced serviceability, accessibility, and improved maintenance activities. In some forms, the components may be operatively coupled using quick-connect devices. For example, in one non-limiting example, the quick connection 109 can be provided in the form of a quick disconnect coupled to both the liquid line inlet 110 and the vapor return line 112, such that the probe assembly 102 and/or hose assembly 111 can be quickly switched out without needing an entire back-up of the closed-loop system.

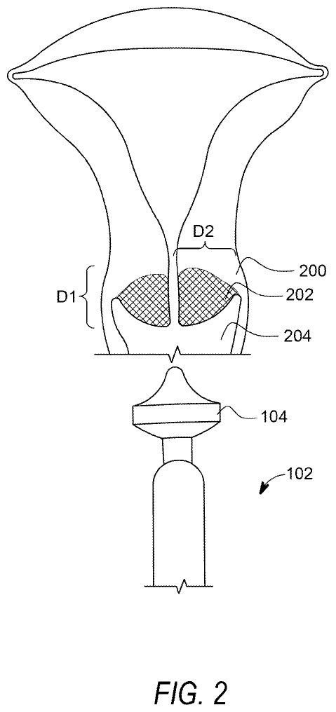

FIG. 2 illustrates a cross-sectional view of a cervix 200, with the probe tip 104 in proximity to a cervical abnormality 202 (e.g., a precancerous lesion or other abnormal cervical tissue). In an exemplary embodiment, the closed-loop cryotherapy system 100 is configured for focused cooling of cervical tissue by applying the cervix-contacting probe tip 104 to a cervical abnormality 202 until a desired freeze zone is achieved. In some embodiments, the desired freeze zone includes a freeze depth D1 and/or a freeze radius D2 of at least 5 mm of tissue at a temperature at least as low as −20° C. for at least one minute. In at least this way, the system can produce a sufficiently cold temperature at a desired freeze depth for effective cryoablation of precancerous lesions and other cervical tissue anomalies. It will be appreciated that various freeze depths D1, freeze radii D2, freeze temperatures, and freeze times can be achieved by the system and processes of the present disclosure. In exemplary embodiments, the probe tip 104 is applied to the cervical tissue in a 5-4-5 double freeze treatment cycle (i.e., five minutes of freeze, four minutes of thaw, and five more minutes of freeze).

In some embodiments, the probe assembly 102, including the probe tip 104, is sized to effectively achieve the desired freeze zone on the cervical abnormality 202, but not to freeze, damage, or otherwise interfere with the tissue of the vaginal wall 204. In one embodiment, the probe tip is within a range of 17 mm-23 mm. In a non-limiting exemplary embodiment, the probe tip is approximately 20 mm. In an exemplary embodiment, the probe tip 104 is provided in the form of a biocompatible metallic tip designed to coordinate with a patient's cervical anatomy, maximize the reach and contact area with the cervical tissue, and minimize patient discomfort. In at least one embodiment, only a portion of the probe tip 104 (e.g., the foremost apex of the probe tip 104) reaches the cryogenic freezing temperatures to minimize incidental contact between a patient's vaginal wall and the freezing probe tip 104.

FIG. 3 illustrates a cross-section of an example of a probe assembly 102 to provide focused cooling and treatment of cervical abnormalities, wherein the probe assembly 102 includes a liquid line inlet 110 which carries cryogenic materials into the probe assembly 102 from the cryogenic circulating unit 115. In this embodiment, the cryogenic material is in a liquid form as it passes through the inlet 305 and travels through evaporating coils 310 in the handle 108 of the probe assembly 102 to the vapor return line 112, wherein the cryogenic material is transferred back to the cryogenic circulating unit 115 in the form of a vapor. As the cryogenic material passes to the probe tip via the liquid line inlet 110 through the evaporator coils and/or the evaporator chamber internal of the internal probe body within the probe sheath 106 to the probe tip 104 (see FIGS. 4A-7B), the probe tip 104 freezes to reach the predetermined cryogenic temperature. According to some embodiments, when the probe tip 104 is applied to the tissue abnormality, the heat from the cervix (or other tissue) is extracted via the probe tip 104 and is transferred through the evaporator chamber of the internal probe body to the vapor return line 112, resulting in freezing of the tissue anomalies and recycling of the cryogenic materials. In some embodiments, the liquid line inlet 110 is provided in the form of a capillary tube, which also serves as a metering device to lower the pressure of the subcooled cryogenic materials from the condenser unit 120 into the probe assembly 102. In some embodiments, the capillary tube is a polyether ether ketone (PEEK) capillary, a copper capillary, an aluminum capillary, or other material. In particular embodiments, the system can include a separate metering device provided in the form of a thermostatic expansion valve. In some forms, the expansion valve is located in line with the liquid inlet line capillary tube to generate asymmetric pressure. In various embodiments, the capillary tube can be a plurality of diameters and lengths. In some embodiments, the capillary tube is sized relative to one or more of the diameter of the evaporation chamber and/or the return vapor line to generate a specific freezing temperature and/or freezing speed. In various embodiments, the ratio of the inner diameter of the capillary tube to the inner diameter of the return vapor line is between the range of 1/30 to 1/2, although other ratios, including ratios smaller than 1/30 and larger than 1/2 are contemplated within the scope of the present disclosure. In some embodiments, the capillary tube can terminate at the proximal end of the probe assembly 102. In some embodiments, including those shown in FIGS. 4A-7B, the terminal end of the capillary tube can be located in the probe tip 104. In some embodiments, a quick connection 109 can be provided at the base of the handle 108, at the proximal end of the probe assembly 102, to disconnect the hose 113 housing the liquid line inlet 110 and the vapor return line 112 for efficient swapping of probe assemblies 102. Alternative configurations of the probe assembly 102 are shown in FIGS. 4A-7B, although these are only exemplary embodiments and additional designs and configurations are considered within the scope of the present disclosure.

FIGS. 4A-7B illustrate cross-sections of examples of a probe assembly 102 according to some embodiments. In some embodiments, the probe assembly 102 of each of the FIGS. 4A-4F have various configurations of an internal probe tip 402a-f, an internal probe body 404a-f, and a probe inlet 406a-f, respectively. For each embodiment, the internal probe tip 402a-f is located within the probe tip 104 at the distal end of the probe assembly. For each embodiment, the internal probe body 404a-f is located within the probe sheath 106. At least in one embodiment, the internal probe body 404a-f is constructed of metal and brazed to the probe tip 104 and coupled to the handle 108 with fittings (not shown). In some embodiments, the probe sheath 106 is constructed of a durable plastic. For each embodiment, the probe inlet 406a-f is located at the proximate end of the probe assembly 102 and includes a connection to both the liquid line inlet 110 and the vapor return line 112. The figures are exemplary, and it will be appreciated that the probe assembly 102 in various embodiments can include one or more aspects of any of the configurations shown in FIGS. 4A-7B. For example, one embodiment of the probe assembly 102 can be provided in the form of the configuration of the probe inlet 406a shown in FIG. 4A with the internal probe body 404b of FIG. 4B and the internal probe tip 402d of FIG. 4D. The materials illustrated in the FIGS. 4A-7B are also exemplary. It will be appreciated that each component can be comprised of a number of various materials and the components herein, despite the hatching shown, can be provided in the form of the same material, different materials, or a combination thereof.

FIG. 4A illustrates an exemplary probe assembly 102 according to one embodiment, wherein the liquid line inlet 110 capillary extends through the center of the probe inlet 406a and extends through the center of the internal probe body 404a. In this embodiment, the outer diameter of the internal probe body 404a is slightly smaller than the inner diameter of the outer probe sheath 106, whereby the diameter of the evaporation chamber 315 (located within the internal probe body 404a impacts the freeze time, temperature, and efficiency of the probe assembly 102. In this example, the liquid line inlet 110 does not extend to the probe tip 104, and the internal probe body 404a terminates at the internal probe tip 402a.

FIG. 4B illustrates an exemplary probe assembly 102 according to one embodiment, wherein the liquid line inlet 110 capillary extends through the center of the probe inlet 406b, and the inner diameter of the internal probe body 404b and the evaporation chamber 315 is significantly smaller than the embodiment shown in FIG. 4A. In the example shown in FIG. 4B, the internal probe body 404b terminates at the internal probe tip 402b and is surrounded by conductive material at the internal probe tip 402b.

FIG. 4C illustrates an exemplary probe assembly 102 according to one embodiment, wherein the liquid line inlet 110 capillary extends through the center of the probe inlet 406c, and the probe inlet 406c is provided in the form of a conical shape for modularity. In this embodiment, the outer diameter of the internal probe body 404c is approximately equal to the inner diameter of the probe sheath 106. The evaporation channel 315 narrows at the internal probe tip 402c to increase the evaporation rate near the probe tip 104. In the example shown in FIG. 4C, the internal probe tip 402c is designed with multiple conductive components to target focused cooling at the probe tip 104.

FIG. 4D illustrates an exemplary probe assembly 102 according to one embodiment, wherein the liquid line inlet 110 capillary extends through the center of the probe inlet 406d and terminates at the proximal end of the internal probe tip 402d. In this embodiment, there is a small gap providing an evaporator channel 315 along the length of the internal probe body 404d and the probe sheath 106. The internal probe tip 402d is designed with an evaporator coil 405d, which coils vertically, perpendicular to the length of the probe assembly 102. The evaporator coil 405d can improve the cooling efficiency of the probe tip 104 and help the probe tip 104 maintain colder temperatures with less heat dissipation.

FIG. 4E illustrates an exemplary probe assembly 102 according to one embodiment, wherein the liquid line inlet 110 capillary enters through the center of the probe inlet 406e but transitions to extend towards the distal end of the probe assembly on a first side of the internal probe body 404e. The inner diameter of the probe sheath 106 is slightly less than the outer diameter of the internal probe body 404e. A portion of the probe inlet 406e extends towards the distal end of the probe assembly 102 and extends into the internal probe body 404e. In this embodiment, the internal probe tip 402e is designed with an evaporator coil 405e, which coils outward from the probe tip 104 in a starburst shape, wherein the coil 405e begins at the termination point of the liquid line inlet 110 and the coil 405e ends near the upper edge of the internal probe body 404e, opposite the side of the liquid line inlet 110.

FIG. 4F illustrates an exemplary probe assembly 102 according to one embodiment, wherein the liquid line inlet 110 capillary enters through the center of the probe inlet 406f but transitions to extend towards the distal end of the probe assembly on a first side of the internal probe body 404f. The proximal side of the internal probe body 404f includes a coupling 415, which contains both the liquid line inlet 110 and a return vapor return line 112. In some embodiments, the coupling 415 can be provided in the form of a quick disconnect for quickly disconnecting and reconnecting the vapor return line 112 and the liquid line inlet 110. The example of FIG. 4F has a probe sheath 106 with an internal diameter approximately equal the outside diameter of the internal probe body 404f. In this example, the liquid line inlet 110 extends through the internal probe body 404f and terminates at the probe tip 104. In this embodiment, the internal probe tip 402f is designed to be filled with cryogenic material in the form of liquid to increase the freezing efficiency of the probe tip.

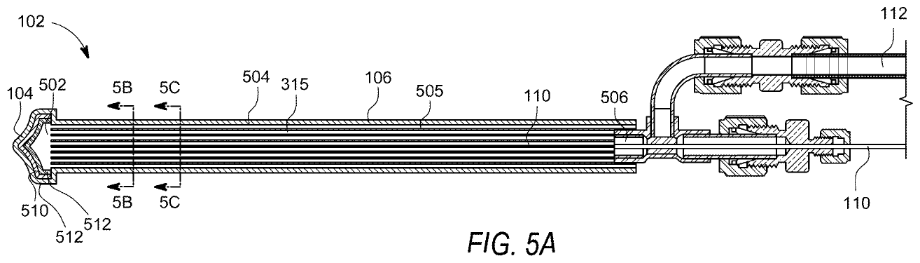

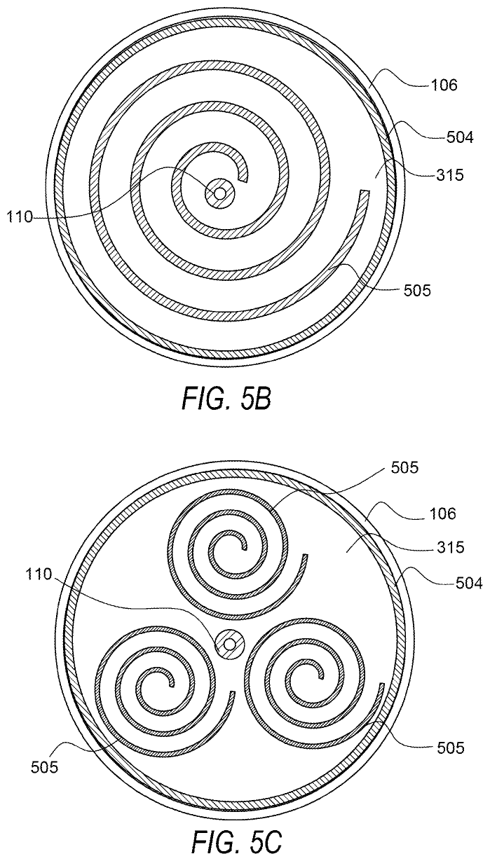

FIG. 5A illustrates an exemplary probe assembly 102 according to one embodiment, wherein the internal probe body 504 comprises one or more thin-rolled copper sheets 505. In some embodiments, the thin-rolled copper sheets 505 are provided in the form of one large spiral around the liquid line inlet 110 (see cross-section in FIG. 5B). In an alternative embodiment, the one or more thin-rolled copper sheets 505 can be coiled around itself and positioned surrounding the inlet on the interior of the internal probe body 504 (see cross-section in FIG. 5C). In this example, the space surrounding the rolled copper sheets 505 provides the evaporation chamber 315 for carrying the cryogenic material in the form of a vapor back to the cryogenic circulating unit 115. It will be appreciated that while copper sheets are used in the example embodiment described herein, other conducting materials can be used in some embodiments. It will also be appreciated that while two examples of the configuration of the thin-rolled copper sheets 505 are provided, alternative configurations and quantities of thin-rolled copper sheets and/or coils can be used. Returning to the embodiment shown in FIG. 5A, the liquid line inlet 110 capillary enters through the center of the probe inlet 506, at the center of the thin-rolled copper sheets 505, and extends towards the distal end of the probe assembly in the center of the internal probe body 504 within the probe sheath 106 and terminates at the proximal end of the internal probe tip 502. In some embodiments, the liquid line inlet 110 is provided in the form of a PEEK liquid line capillary. In some embodiments, the liquid line inlet 110 is provided in the form of a copper liquid line capillary. In this embodiment, the internal probe tip 502 is designed to be filled with a cryogenic material provided in the form of a freezable gel 510 to increase the freezing efficiency of the probe tip 104. In some embodiments, the gel is one or more of polypropylene glycol, sodium polyacrylate, refrigerant oil, dimethicone fluid, calcium chloride solution, or similar freezable gel material. In the example shown in FIG. 5A, the freezable gel 510 is positioned between two probe walls 512, wherein the outer probe wall 512 can be the probe tip 104. In one non-limiting embodiment, the probe walls 512 are constructed of copper. In one non-limiting embodiment, the probe sheath 106 is constructed of copper, steel, ceramic, or a combination thereof. In one non-limiting embodiment, the probe sheath 106 has a double-walled construction.

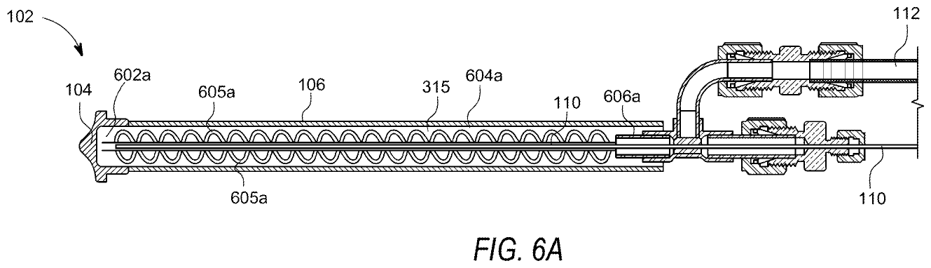

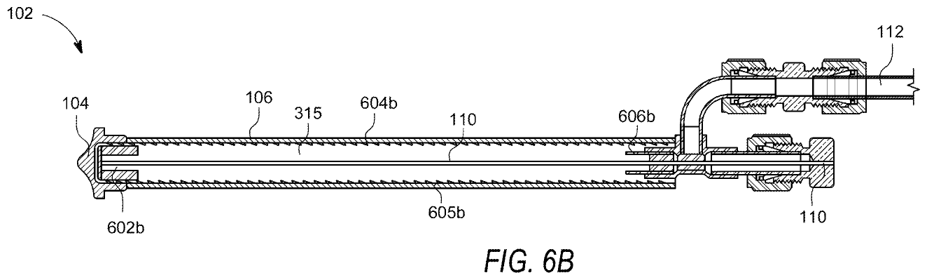

FIG. 6A illustrates an exemplary probe assembly 102 according to one embodiment, wherein the internal probe body 604a comprises one or more propellers or fan-like fins 605a, which cool the cryogenic material through the evaporator channel 315 to further increase cooling. In some embodiments, the fan-like fins 605 spiral around the liquid line inlet 110 along the length of the probe sheath 106, wherein the liquid line inlet 110 is centered from the probe inlet 606a through the internal probe body 604a within the probe sheath 106 to the termination point for the liquid line inlet 110 at the internal probe tip 602a.

FIG. 6B illustrates an exemplary probe assembly 102 according to one embodiment, wherein the internal probe body 604b comprises one or more threaded ends 605b. In some embodiments, the internal probe body 604b is provided in the form of a threaded rod, which inherently creates the one or more threaded ends 605b. In one non-limiting embodiment, the internal probe body 604b is provided in the form of a brass threaded rod, and the liquid line inlet 110 extends through the center of a hole in the center of the brass threaded rod. In the example embodiment shown in FIG. 6B, the liquid line inlet 110 enters the probe assembly 102 at the center of the probe inlet 606b. In this example, the liquid line inlet 110 can extend the length of the probe sheath 106 and terminate at the internal probe tip 602b. It will be appreciated that while brass is used in the example embodiment described herein, other threaded materials can be used in some embodiments (e.g., steel, a double-threaded hollow bolt instead of a hollow rod, etc.). In the embodiments shown in FIGS. 6A and 6B, the internal probe assembly 102 geometries are designed to impede the flow of the cryogenic materials through the probe sheath 106, which increases the contact duration and surface area between the evaporating cryogenic material and the probe wall material, which improves heat transfer capabilities of the probe assembly 102 and generates faster freezing techniques.

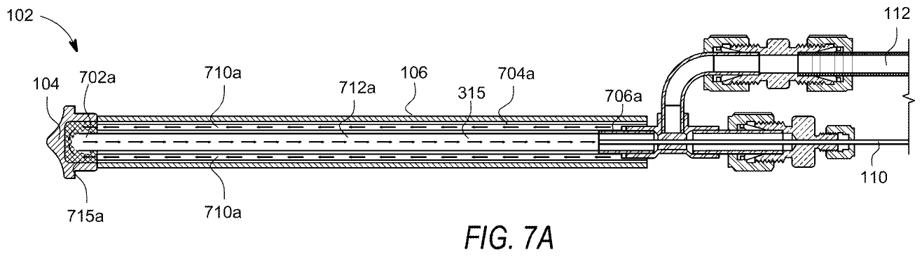

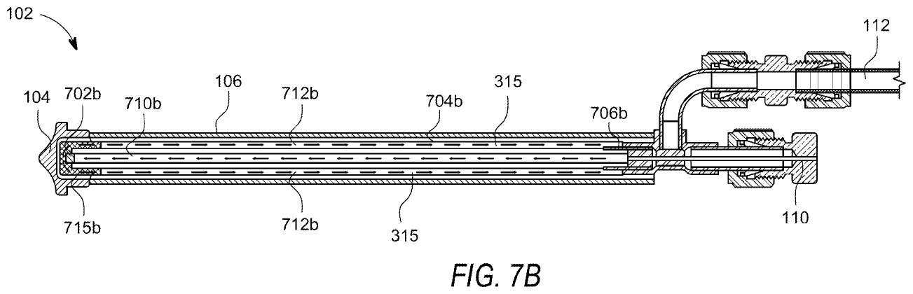

FIGS. 7A and 7B illustrate alternative embodiments of an exemplary probe assembly 102 configured for a reverse flow process to provide faster freezing since cooling from the vapor return line 112 cools the cryogenic material in the liquid line inlet 110 as it travels towards the probe tip. In some embodiments, the internal probe tip 702a and 702b, respectively, include a sintered filter 715a and 715b to separate particulate matter from the cryogenic material and more evenly distribute the cryogenic material across the probe tip 104. The reverse flow configurations shown in FIGS. 7A and 7B are only exemplary, and it will be appreciated that alternative arrangements of the liquid line inlet 110 and vapor return line 112 and corresponding internal chambers for each are considered within the scope of the present disclosure.

In FIG. 7A, the liquid line inlet 110 enters the probe inlet 706a centered, and at an overlap between the probe inlet 706 and the internal probe body 704a, the inlet chambers 710a are routed along the length probe sheath 106 on either side of the outlet chamber 712a, which is centered along the length of the probe sheath 106 in the internal probe body 704a until the termination point at the internal probe tip 702a.

In FIG. 7B, the liquid line inlet 110 enters the probe inlet 706b centered and at an overlap between the probe inlet 706 and the internal probe body 704b, the liquid line inlet 110 transfers to an inlet chamber 710b with an increased diameter compared to the liquid line inlet 110 capillary. The outlet chambers 712b are routed along the length probe sheath 106 on either side of the inlet chamber 710a, which is centered along the length of the probe sheath 106 in the internal probe body 704b until the termination point at the internal probe tip 702b.

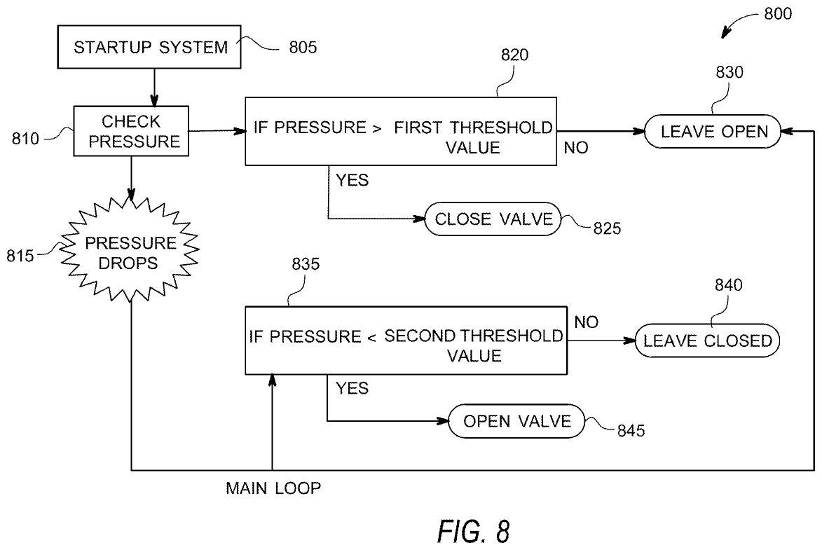

FIG. 8 is an illustrative overview of a throttling process 800 to increase the high side and low side pressure differential (producing colder temperatures) by modulating the flow of the cryogenic materials to maintain optimal low side pressure, wherein the low side pressure is based on the pressure of the vapor return line 112 on the suction side of the compressor 114 and the high side pressure is based on the pressure reading at the output of the condenser unit 120. At step 805, the throttling system starts and a pressure sensor determines the pressure of the system at step 810. In some embodiments, the system utilizes a temperature sensor to determine when the probe assembly 102 has reached a stable temperature before initiating the throttling system at step 805. In some embodiments, the system can utilize a timer before starting the throttling system at step 805. The controller 128 (or other system component) determines if the pressure is greater than the first threshold value at step 820. In one non-limiting embodiment, the first threshold pressure is approximately 18 psi. If yes, the controller 128 (or other system component) closes a throttling expansion valve (not shown) at step 825. If the detected pressure is less than the first threshold value at step 820, the controller 128 (or other system component) leaves the throttling expansion valve open at step 830. The system 100 monitors the pressure of and determines if there is a pressure drop in the main loop of the cryogenic circulating unit 115 at step 815. The controller 128 (or other system component) determines if the detected pressure is less than a second threshold value at step 835. In one non-limiting embodiment, the second threshold value is approximately 13 psi. If yes, the controller 128 (or other system component) opens the throttling expansion valve at step 845. If the detected pressure is greater than the second threshold value at step 835, the controller 128 (or other system component) leaves the throttling expansion valve closed at step 840. In at least this way, the opening and closing of the throttling expansion valve according to the pressure reading relative to the first and second threshold value can facilitate an optimal pressure differential between the high side and low side of the closed-loop system.

FIG. 9 illustrates a non-limiting exemplary embodiment of a medical device 900 comprising the closed-loop cryogenic system 100. In this embodiment, the cryogenic circulating unit 115 is enclosed within a portable durable housing 905 with one or more handles 910, one or more wheels 915, a control device 920, and a display screen 925. The housing 905 further includes a hose 930 to connect the probe assembly 102 to the housing 905 via a connection port 940 and a hose connector 945. Where used herein, “portable” can include various aspects of transportability including the ability to relocate the system between exam rooms, clinic locations, remote areas, and international travel. Where used herein, “durable” can include various aspects of reliability including materials capable of withstanding harsh operating and environmental conditions (uneven surfaces, dust, dirt, water, impact, corrosion). The housing 908 (and all the system components) is also designed to be easily cleaned, sanitized, and maintained. In this example, ‘clean” can include the physical removal of contaminants (e.g., dirt, dried blood, etc.), and “sanitize” can include disinfection and the removal/killing of microorganisms. In some embodiments, the housing 905 is constructed out of a durable thermoplastic material mounted to a metal base, wherein the cryogenic circulating unit 115 enclosed within the housing 905 is a fully brazed system. It will be appreciated that other materials can be used for the construction of the housing 905 within the scope of the present disclosure. In some embodiments, aspects of the probe assembly 102 can be designed to be easily cleaned and sanitized (e.g., a removable and exchangeable probe tip 104 and/or probe sheath 106).

To aid in portability, the housing 905 can include one or more movable wheels 915. In some embodiments, the wheels 915 can be provided in the form of caster, tracks, or similar transport components to allow the device 900 to be moved from one location to another without physically lifting the device 900 off the ground. In various embodiments, the wheels 915 can be lockable to prevent the device 900 from moving during a treatment. The device 900 can also include one or more handles 910. In some embodiments, like the example shown in FIG. 9, the handle 910 can be provided in the form of an inset handle. In various embodiments, the handle 910 can also be various shapes, sizes, and configurations (e.g., swooped, rectangle, rounded, standard, etc.) to provide a grasping mechanism for a user to move the device 900. In some embodiments, the housing 905 can include multiple handles 910. In some embodiments, the housing 905 can include one or more removably coupled straps (not shown) to provide another means for transporting the device 900 via a shoulder if traveling via motorcycle, for example. In some embodiments, the housing 905 is designed to minimize the ability of the device 900 to tip over, which can include configuring the shape and dimensions of the housing 905 and/or the weight distribution of the cryogenic circulating unit 115 within the housing 905. In some embodiments, the top side 950 of the housing 905 can be designed (e.g., with flat, non-slip, raised lips) to facilitate holding procedural equipment and materials, or stacking other materials when the device is not in use. Some embodiments can also include a hose rack (not shown) to neatly store the hose 930 for the probe assembly 102. In some embodiments, the probe assembly 102 can be pre-charged with a refrigerant amount or weight, such that a service provider could quickly disconnect a used probe and connect a new pre-charged probe for the efficient treatment of multiple patients.

In some embodiments, the control device 920 can be provided in the form of a single push button and/or movable controller (e.g., joystick) and/or a keypad for one or more inputs. In some embodiments, the control device 920 can include an externally connected device like a remote control, laptop, tablet, mobile device, or similar device capable of receiving and processing user input.

In some embodiments, the display screen 925 can be provided in the form of a touch screen device capable of receiving user input and displaying information to the user/service provider. In one non-limiting example, the display screen 925 can be used to generate one or more notifications from the notification module (not shown). Non-limiting examples of the one or more notifications can include a system status (e.g., OFF, ON, CHARGING, TREATMENT IN PROGRESS, etc.), errors (e.g., LOW BATTERY, LOW PRESSURE, HIGH PRESSURE, etc.), and treatment information (e.g., FREEZE, THAW, SECOND FREEZE, TIME REMAINING, TREATMENTS REMAINING, etc.). In various embodiments, the display screen 925 can also be used to display maintenance reminders, cleaning instructions, sanitization instructions, troubleshooting tips, diagnostic information, advanced analytics collected by the controller 128, or other data. In some embodiments, the control device 920 can be used to customize the display outputs on the display screen 925.

In various embodiments, the housing is designed with an advanced ventilation system including one or more vents 955. In some embodiments, the vents can be provided in the form of slits on one or more sides of the housing 905. In some embodiments, the system 100 can include an exhaust fan (not shown) or similar active ventilation component. In this example, a temperature sensor and the controller 128 can be used to activate the exhaust fan when an internal housing temperature reaches a certain threshold value. In one non-limiting embodiment, the cryogenic circulating unit 115 is water-cooled (e.g., a volume of cool water is poured into the housing during use) to keep the compressor 114 cool and improve the condenser 120 function. In this example, the water is drained once it warms up and the system is refilled with cool water as needed. In some embodiments, the water-cooled process is automated using the controller 128 and one or more temperature sensors and an actuated drain valve (not shown).

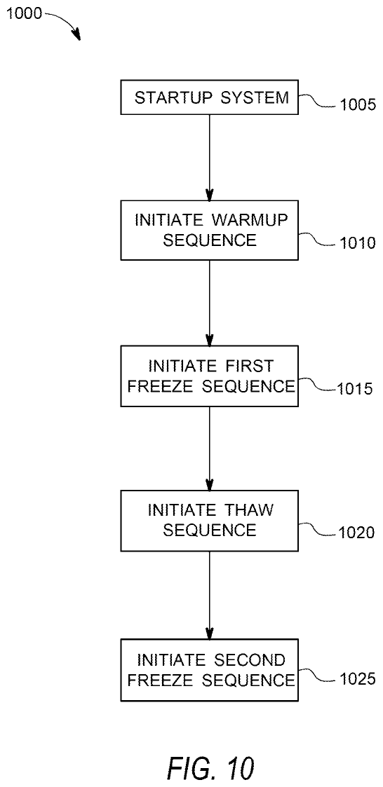

FIG. 10 illustrates a cryotherapy process 1000 for activating a double freeze treatment cycle according to some embodiments. At step 1005, the system is powered on and cryogenic materials begin to cycle through the closed-loop system 100. At step 1010, a warmup sequence is initiated. In some embodiments, the warmup sequence runs for a predetermined amount of time based on one or more timer circuits, or until the probe tip 104 reaches a specified temperature within a predefined time period (e.g., the probe tip 104 reaches −20° C. within 20 minutes). In this non-limiting example, if the probe tip 104 reaches the specified temperature within a predefined time period, the system can automatically advance to step 1015. In some embodiments, the warmup sequence runs until the probe assembly 102 reaches a stable temperature and/or pressure. After the warmup sequence is complete, the first freeze sequence is initiated at step 1015. In a non-limiting example, the condenser unit 120 cools the cryogenic material, which is then carried to the probe assembly 102 for focused freezing of the probe tip 104. In one non-limiting embodiment, the first freeze sequence runs for five minutes, based on a timer or a temperature reading. In some embodiments, the timer for the first freeze sequence is not activated until the temperature of the probe tip 104 is equal to the predetermined treatment temperature. In exemplary embodiments, the treatment temperature is between at least −20° C. with an optimal treatment temperature range of approximately −30° C. to −80° C. It will be appreciated that the predetermined treatment temperature described herein is based on cryotherapy applications for treating cervical abnormalities and other treatment temperatures can be achieved with the system and processes described herein. At step 1020, the system initiates a thaw sequence. In one non-limiting embodiment, the thaw sequence runs for 4 minutes, based on a timer or based on a temperature reading. According to some embodiments, the system is powered off or otherwise in a sleep mode during the thaw sequence and the system does not actively cool or circulate the cryogenic material. In some embodiments, the system can include a parallel path to actively flow the cryogenic material away from the probe assembly 102 during the thaw sequence. At step 1025, the system initiates a second freeze sequence. In one non-limiting embodiment, the second freeze sequence runs for 5 minutes, based on a timer or a temperature reading. In some embodiments, the timer for the second freeze sequence is not activated until the temperature of the probe tip 104 is equal to the predetermined treatment temperature. It will be appreciated that other configurations of the freeze cycle, including for various freeze and thaw times, are contemplated within the scope of the present disclosure.

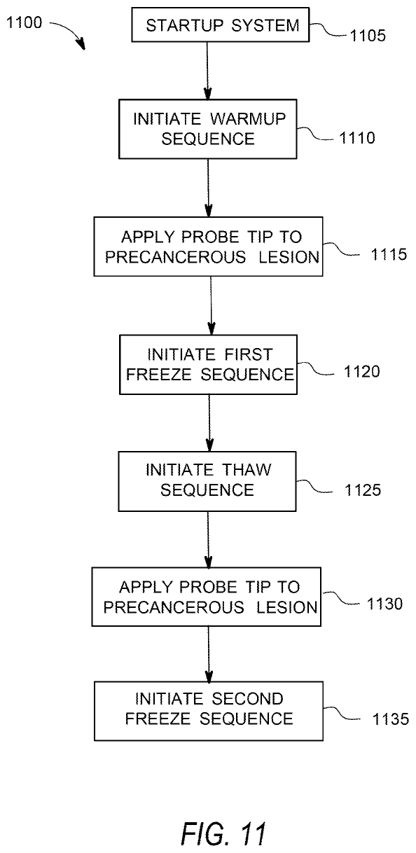

FIG. 11 illustrates a cryoablation process 1100 for applying a double freeze treatment cycle to treat a precancerous lesion, according to some embodiments. At step 1105, a service provider verifies a probe assembly 102 is plugged into the probe connection port 940 of the device 900 and starts up the system using the control device 920 on the housing 905 (see FIG. 9). In at least one embodiment, the cryogenic circulating unit 115 can be powered on and operate when the front-end subsystem 105 is not connected. At step 1105, cryogenic materials begin to cycle through the closed-loop system 100. At step 1010, a warmup sequence is initiated. In some embodiments, the warmup sequence runs for a predetermined amount of time based on one or more timer circuits or a temperature reading. In one non-limiting example, the display screen 925 of the housing 905 can display a timer and notify the service provider when the system switches between the warmup sequence and the first freeze sequence at step 1115. In some embodiments, the warmup sequence runs until the probe assembly 102 reaches a stable temperature and/or pressure. In this example, the display screen 925 of the housing 905 can display a notification to the service provider when the probe assembly 102 has reached a stable temperature and/or pressure. After the warmup sequence is complete, the service provider applies the probe tip 104 to the cervical tissue 200 (see FIG. 2) at step 1115, and a first freeze sequence is initiated at step 1120. The probe tip 104 should be applied directly to the tissue abnormality 202 while avoiding the vaginal walls, to the extent possible. In one non-limiting embodiment, the first freeze sequence runs for five minutes, based on a timer or a temperature reading. In some embodiments, a timer for the first freeze sequence does not activate until the temperature of the probe tip 104 is equal to the predetermined treatment temperature. In exemplary embodiments, the treatment temperature is between at least −20° C. with an optimal treatment temperature range of approximately −30° C. to −80° C. In one non-limiting example, the treatment temperature is approximately −50° C. It will be appreciated that the predetermined treatment temperature described herein is based on cryotherapy applications for treating cervical abnormalities and other treatment temperatures can be achieved with the system and processes described herein. In this example, the display screen 925 of the housing 905 can generate a notification that the system is in “FIRST FREEZE SEQUENCE” mode with the remaining time displayed based on the output of the timer circuit(s). At step 1125, the system initiates a thaw sequence and the service provider maintains contact with the cervical tissue during the thaw cycle. In some embodiments, the service provider can withdraw the probe tip from the cervix during the thaw cycle. In one non-limiting embodiment, the thaw sequence runs for 4 minutes, based on a timer or a temperature reading. In this example, the display screen 925 of the housing 905 can generate a notification that the system is in “THAW” mode with the remaining time displayed based on the output of the timer circuit(s). At step 1130 the service provider reapplies or maintains contact between the probe tip 104 and the cervical tissue 200 and the system initiates a second freeze sequence at step 1130. In one non-limiting embodiment, the second freeze sequence runs for 5 minutes, based on a timer or a temperature reading. In some embodiments, the timer for the second freeze sequence will not be activated until the temperature of the probe tip 104 is equal to the predetermined treatment temperature. In this example, the display screen 925 of the housing 905 can generate a notification that the system is in “SECOND FREEZE SEQUENCE” mode with the remaining time displayed based on the output of the timer circuit(s). The service provider shall remove the probe assembly 102 from the vaginal canal after the conclusion of the second freeze sequence. It will be appreciated that other configurations of the freeze cycle, including for various freeze and thaw times, are contemplated within the scope of the present disclosure.

In some embodiments, the system can provide a haptic, visual, or auditory notification, or similar (e.g., vibration, indicator light, chime, etc.) in lieu of a notification displayed on the display screen (or a push notification to a user's mobile device, or similar) to indicate different modes of operation or phases of treatment. In some embodiments, the service provider can follow written instructions related to the procedure steps in lieu of being prompted to move on to the next phase of treatment by the system display.

In one embodiment, the present disclosure provides a closed-loop cryotherapy system in the form of a portable, battery-powered, medical device designed for low-resource medical settings to provide a comprehensive standard-of-care solution for cervical precancer treatment by freezing cervical tissue abnormalities, including precancerous lesions. This embodiment includes a front-end probe assembly and a backend subsystem comprising a cryogenic circulating unit enclosed within a durable and ventilated housing, wherein the housing includes at least one handle and a set of wheels to move the device between rooms of a medical setting. In this embodiment (and others), the front-end probe assembly includes a probe tip with an internal probe tip, a probe sheath with an internal probe body, and an ergonomic handle that contains a probe inlet. In various embodiments, the probe inlet is coupled to a hose assembly, wherein the hose assembly includes a liquid line inlet and an outlet vapor return line, such that liquid cryogenic materials enter the probe, travel through a chamber within the internal probe body to freeze the probe tip, then the vaporized cryogenic materials are recycled to the backend subsystem via the vapor return line outlet. In this embodiment, the cryogenic materials are provided in the form of a refrigerant (in the form of vapor, liquid, gas, and/or gel) that has a low GWP, have zero ODP, is suitable for low and back middle back pressure compressor system, and has a dew point or melting point below 10° C. at atmospheric pressure. The hose assembly can be provided in the form of a durable polymer sheathing, multi-lumen polymer tubing, or similar tubing. In this embodiment, the liquid line inlet is provided in the form of a PEEK tubing, and the vapor return outlet is provided in the form of a refrigerant-rated polymer hose. The front-end probe assembly can also be configured with a quick disconnect at the proximate end of the probe assembly and/or within the hose assembly to allow a service provider to quickly disconnect the probe and reconnect a new probe assembly and the quick disconnect is configured to seal to prevent air or other environmental contaminants to enter the system. The backend cryogenic circulating unit can include a single-stage compressor with variable speed efficiency and a displacement of approximately 2.0 cm³ to 3.5 cm³ or a cooling capacity of approximately 50 Watts to 830 Watts, an oil-separator loop with a coalescent oil separator, a tube, and a solenoid, a condenser unit with aluminum microchannel serpentine coils, a fan with variable speed efficiency and an operating range of approximately 30 to 300 CFM, a controller, a power supply provided in the form of a LiFePO₄ battery, and a solid sieve core copper filter drier. The cryogenic circulating unit recycles the cryogenic materials used to freeze the probe tip to eliminate the need for venting gases to the environment or using consumable gases to operate the system. Further, since the power supply can be provided in the form of a rechargeable battery to allow the system to operate without a stable power supply.

Some embodiments of the system include a test fixture design with a cervical tissue benchtop model design and mold for testing, troubleshooting, and system parameter verification. Various embodiments can include a sous vide setup, thermocouple placement fixture, and others. In some embodiments, the cervical tissue benchtop mold can include various geometries (e.g., a conical anatomical shape, a flat simplified design, etc.) and materials (e.g., agar, ballistic gel, calcium phosphate solution). In some embodiments, the configuration of the test molds are designed to facilitate a visualization of the freeze zone.

While the particular embodiments described herein relate to medical-based cervical treatment applications, it will be recognized and appreciated that the systems and processes described herein are also applicable to at least, but not including, other types of freezing, cooling, or heating applications. For example, the systems and methods described herein could be used for other medical solutions related to cryotherapy, cryopreservation, cryosurgery, cryostats, dermatology, ENT, gynecology, general surgery, oncology, etc. According to some dermatology-related embodiments, the system can be used for the ablation or freezing of skin cancers and other cutaneous disorders, the palliation of tumors of the skin, and/or the destruction of warts or lesions. In various additional gynecology applications, the system can be used for the ablation of malignant neoplasia or benign dysplasia of the female genitalia In various ENT-related applications, the system can be used for palliation of tumors of the oral cavity and/or the ablation of leukoplakia of the mouth. In various general surgery applications, the system can be used for the ablation of leukoplakia of the mouth, angiomas, sebaceous hyperplasia, dermatofibromas, small hemangiomas, mucocele cysts, multiple warts, plantar warts, hemorrhoids, anal fissures, perianal condylomata, pilonidal cysts actinic and seborrheic keratoses, cavernous hemangiomas, recurrent cancerous lesions, palliation of tumors of the rectum, hemorrhoids, anal fissures, pilonidal cysts, recurrent cancerous lesions, destruction of warts or lesions, palliation of tumors of the oral cavity, rectum, skin, etc. In some embodiments, the system can be used to destroy tissue by the application of extreme cold temperatures including prostate and kidney issues, liver metastases, tumors, skin lesions, and warts. In some embodiments, the system can be used with an imaging device, like an ultrasound, to provide real-time visualization of the cryosurgical procedure. Additionally, the systems and methods described herein could be used for food and beverage applications like rapid creation of ice for whiskies and other libations, processing, preparation, preservation, presentation, flash freezing, blast freezing, quick freezing, etc. Further, the system and techniques described herein could be applied in research and development settings for efficient freezing to test material thresholds and operating parameters under various freezing conditions. In some embodiments, the system can include a heat pipe designed to heat the probe tip 104 to provide thermal ablation to treat tissue abnormalities.

It will be appreciated by one skilled in the art that this embodiment is only a non-limiting example used to illustrate that the system and processes are designed to dynamically provide a closed-loop cryotherapy system to recycle and circulate cryogenic materials, wherein a liquid form of the cryogenic material expands within an evaporation chamber of a probe assembly to quickly generate freezing temperatures focused at a tip of the probe assembly. Additionally, the non-limiting examples can be used for focused cooling and heating across a plurality of systems and applications and can apply to a plurality of different services and industries.

Computer program code that implements the functionality described herein typically comprises one or more program modules that may be stored on a data storage device. This program code, as is known to those skilled in the art, usually includes an operating system, one or more application programs, other program modules, and program data. A user may enter commands and information into ta the computer through a keyboard, touch screen, pointing device, a script containing computer program code written in a scripting language, or other input devices, such as a microphone, etc. These and other input devices are often connected to the processing unit through known electrical, optical, or wireless connections.

The computer that affects many aspects of the described processes will typically operate in a networked environment using logical connections to one or more remote computers or data sources, which are described further below. Remote computers may be another personal computer, a server, a router, a network PC, or other common network nodes common network node, and typically include many or all of the elements described above relative to the main computer system in which the systems are embodied. The logical connections between computers include a local area network (LAN), a wide area network (WAN), virtual networks (both WAN or LAN), and wireless LAN (WLAN) that are presented here by way of example and not limitation. Such networking environments are commonplace in office-wide or enterprise-wide computer networks, intranets, and the Internet.

When used in a LAN or WLAN networking environment, a computer system implementing aspects of the system is connected to the local network 1170 through a network interface or adapter. When used in a WAN or WLAN networking environment, the computer may include a modem, a wireless link, or other mechanisms for establishing communications over the WAN, such as the Internet. In a networked environment, program modules depicted relative to the computer, or portions thereof may be stored in a remote data storage device. It will be appreciated that the network connections described or shown are non-limiting examples and other mechanisms of establishing communications over WAN or the Internet may be used.

Additional aspects, features, and processes of the claimed systems will be readily discernible from the description herein, by those of ordinary skill in the art. Many embodiments and adaptations of the disclosure and claimed systems other than those herein described, as well as many variations, modifications, and equivalent arrangements and processes, will be apparent from or reasonably suggested by the disclosure and the description thereof, without departing from the substance or scope of the claims. Furthermore, any sequence(s) and/or temporal order of steps of various processes described and claimed herein are those considered to be the best mode contemplated for carrying out the claimed systems. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in a variety of different sequences and orders, while still falling within the scope of the claimed systems. In addition, some steps may be carried out simultaneously, contemporaneously, or in synchronization with other steps.

Aspects, features, and benefits of the claimed devices and processes for using the same will become apparent from the information disclosed in the exhibits and the other applications as incorporated by reference. Variations and modifications to the disclosed systems and processes may be affected without departing from the spirit and scope of the novel concepts of the disclosure.

It will, nevertheless, be understood that no limitation of the scope of the disclosure is intended by the information disclosed in the exhibits or the applications incorporated by reference; any alterations and further modifications of the described or illustrated embodiments, and any further applications of the principles of the disclosure as illustrated therein are contemplated as would normally occur to one skilled in the art to which the disclosure relates.

The description of the disclosed embodiments has been for illustration and description and is not intended to be exhaustive or to limit the devices and processes for using the same to the precise forms disclosed. Many modifications and variations are possible in light of the above teaching.

The embodiments were chosen and described to explain the principles of the devices and processes for using the same and their practical application to enable others skilled in the art to utilize the devices and processes for using the same and various embodiments and with various modifications as are suited to the particular use contemplated. Alternative embodiments will become apparent to those skilled in the art to which the present devices and processes for using the same pertain without departing from their spirit and scope. Accordingly, the scope of the present devices and processes for using the same is defined by the appended claims rather than the description and the embodiments described therein.

What is claimed is:

1. A closed-loop cryotherapy system comprising:
a probe assembly configured for focused cooling, the probe assembly comprising:
a cervix-contacting probe tip at a distal end of the probe;
an internal probe body in fluid connection with the cervix-contacting probe tip and comprising an internal evaporation chamber configured to circulate one or more cryogenic materials;
a cryogenic circulating unit designed to recycle the one or more cryogenic materials, the cryogenic circulating unit comprising:
a compressor configured to receive the one or more cryogenic materials from the probe and pressurize the one or more cryogenic materials;
a condenser unit configured to convert the pressurized one or more cryogenic materials to a liquid, wherein the liquid is circulated to the probe assembly to facilitate freezing of a precancerous lesion;
a power module comprising a rechargeable battery, wherein the power module allows the closed-loop cryotherapy system to operate without a stable power grid;
a controller configured to modulate a speed of the compressor in order to adjust a freezing temperature of the probe, wherein the freezing temperature of the probe assembly is within −30 degrees C. and −80 degrees C., and wherein the controller modulates the speed of the compressor based on an ambient temperature of one or more aspects of the probe assembly; and
one or more sensors configured to determine one or more parameters of the system, wherein the one or more sensors includes a pressure sensor, a temperature sensor, or a combination thereof.

2. (canceled)

3. (canceled)

4. (canceled)

5. (canceled)

6. The closed-loop cryotherapy system of claim 1, wherein the cryogenic circulating unit is coupled to the probe assembly with a coaxial hose comprising a liquid line inlet housed within a vapor return line to facilitate efficient freezing of the cervix-contacting probe tip.

7. The closed-loop cryotherapy system of claim 6, further comprising:
a throttling expansion valve configured to open and close to throttle the controller based on a pressure of the liquid in the probe compared to a setpoint threshold.

8. The closed-loop cryotherapy system of claim 7, further comprising:
a housing configured enclose the closed-loop cryotherapy system to allow the closed-loop cryotherapy system to be portable.

9. The closed-loop cryotherapy system of claim 8, wherein the cryogenic circulating unit operates without the use of consumable gases.

10. A closed-loop cryotherapy process comprising:
providing a handheld probe assembly with a cervix-contacting probe tip configured for focused freezing of a tissue, wherein the probe assembly includes an interior evaporation chamber configured to circulate one or more cryogenic materials;
recycling the one or more cryogenic materials using a cryogenic circulating unit comprising a compressor, a condenser unit, a power module, and a controller by:
receiving the one or more cryogenic materials from the probe assembly;
pressurizing the one or more cryogenic materials using the compressor;
converting the one or more cryogenic materials to a liquid using the condenser unit;
circulating the liquid to the probe using one or more hoses;
decreasing a pressure of the liquid before circulating the liquid to the probe assembly by using an expansion valve in line with the one or more hoses; and
throttling the controller by:
receiving a pressure of the liquid in the probe assembly;
determining whether the pressure is above a threshold setpoint;
closing a throttling expansion valve when the pressure is greater than the threshold setpoint; and
opening the throttling expansion valve when the pressure is less than the threshold setpoint.

11. (canceled)

12. (canceled)

13. The closed-loop cryotherapy process of claim 10, wherein the focused freezing of the tissue comprises freezing the tissue to achieve a tissue freeze radial dimension of approximately 5 mm.

14. The closed-loop cryotherapy process of claim 13, wherein the cryogenic circulating unit is configured to recycle the one or more cryogenic materials rather than venting the one or more cryogenic materials into an environment.

15. A closed-loop cryotherapy system comprising:
a probe assembly configured for freezing cervical tissue, the probe comprising:
a cervix-contacting probe tip at a distal end of the probe assembly;
an internal probe body with an internal evaporation chamber configured to circulate one or more cryogenic materials;
a cryogenic circulating unit designed to capture the one or more cryogenic materials the cryogenic circulating unit comprising:
a compressor configured to receive the one or more cryogenic materials from the probe via a vapor return line and pressurize the one or more cryogenic materials;
an oil separator loop configured to process the one or more cryogenic materials;
a condenser unit configured to convert the one or more cryogenic materials from the oil separator loop to a liquid;
an inlet capillary tube configured to carry the liquid to the cervix-contacting probe tip;
an expansion valve in line with the capillary tube designed to generate an asymmetric pressure;
a power module comprising a battery, wherein the power module allows the closed-loop cryotherapy system to operate without a stable power grid; and
a controller configured to modulate a speed of the compressor in order to adjust a freezing temperature of the probe assembly, wherein the controller modulates the speed of the compressor based on an ambient temperature of one or more aspects of the probe assembly.

16. The closed-loop cryotherapy system of claim 15, wherein the freezing temperature of the cervix-contacting probe tip is within −30 degrees C. and −80 degrees C.

17. The closed-loop cryotherapy system of claim 16, wherein the cervix-contacting probe tip is used to freeze a precancerous lesion by applying the cervix-contacting probe tip to an area of tissue until a tissue freeze radial dimension of 5 mm is achieved.

18. A method for treating precancerous lesions of a cervix using a closed-loop cryotherapy system, the method comprising:
positioning a portable housing containing the closed-loop cryotherapy system near a patient, wherein the closed-loop cryotherapy system is configured with a rechargeable power module to allow the closed-loop cryotherapy system to operate without a stable power grid and a cryogenic circulating unit designed to recycle one or more cryogenic materials to allow the closed-loop cryotherapy system to operate without compressed gas;
initiating focused cooling of a probe assembly of the closed-loop cryotherapy system, wherein the probe assembly comprises a cervix-contacting probe tip at a distal end of the probe assembly;
determining the cervix-contacting probe tip has a temperature within −30 degrees C. and −80 degrees C.; and
applying the cervix-contacting probe tip to the cervix until a tissue freeze radial dimension of 5 mm is achieved.

19. The method for treating precancerous lesions of claim 18, further comprising:
throttling a controller of the closed-loop cryotherapy system by:
receiving a pressure of a liquid in the probe assembly;
determining whether the pressure is above a threshold setpoint;
closing a throttling expansion valve when the pressure is greater than the threshold setpoint; and
opening the throttling expansion valve when the pressure is less than the threshold setpoint.

20. The method for treating precancerous lesions of claim 19, further comprising:
allowing the closed-loop cryotherapy system to operate for at least 10 minutes before applying the cervix-contacting probe tip to the cervix.

21. A refrigeration system comprising:
a probe assembly operatively connected to a cryogenic circulating unit designed to recycle cryogenic material, the probe assembly comprising:
a freezing probe tip at a distal end of the probe assembly; and
an internal probe body in fluid connection with the freezing probe tip and comprising an internal evaporation chamber configured to circulate one or more cryogenic materials, wherein:
the probe assembly receives the one more cryogenic materials in a liquid form from a condenser unit of the cryogenic circulating unit thereby cooling the freezing probe tip to −30° C. to −80° C.;
the freezing probe tip is configured to contact human or animal anatomy and absorb heat therefrom, thereby converting the one or more cryogenic materials into a gas form via the internal evaporation chamber and cooling the human or animal anatomy; and
the probe assembly transfers the one or more cryogenic materials in the gas form to the cryogenic circulating unit for pressurizing, condensing, and recycling the one or more cryogenic materials.

22. A refrigeration system comprising:
a cryogenic circulating unit designed to recycle one or more cryogenic materials comprising:
a power module comprising a rechargeable battery, wherein the power module enables the cryogenic circulating unit to operate without a stable power grid;
a controller configured to modulate a speed of a compressor in order to adjust a temperature of the one or more cryogenic materials;
the compressor configured to receive the one or more cryogenic materials from a probe assembly in a gas form and pressurize the one or more cryogenic materials; and
a condenser unit configured to convert the pressurized one or more cryogenic materials to a liquid, wherein the liquid is circulated to the probe assembly to generate a temperature at a probe tip of the probe assembly of −30° C. to −80° C. to facilitate freezing of a precancerous lesion,
wherein the cryogenic circulating unit operates without the use of consumable gases.
